(19) United States
(12) Patent Application Publication
(10) Pub. No.: US 2026/0078338 A1
(43) Pub. Date: Mar. 19, 2026

(54) HIGH-REACTIVITY HYDROGEL PARTICLE AND PREPARATION METHOD THEREFOR AND USE THEREOF

(71) Applicant: NANJING TECH UNIVERSITY, Nanjing (CN)

(72) Inventors: Ziyi YU, Nanjing (CN); Yunfeng ZENG, Nanjing (CN); Jing ZHANG, Nanjing (CN); Yongyuan HENG, Nanjing (CN)

(21) Appl. No.: 18/992,868

(22) PCT Filed: Oct. 17, 2023
(86) PCT No.: PCT/CN2023/124895

§ 371 Date: Jan. 9, 2025

(30) Foreign Application Priority Data
Sep. 13, 2023 (CN) 202311177672.5

(51) Int. Cl. C12N 5/00; B33Y 70/00; C08J 3/075; C12N 9/26; C12N 9/64; C12N 9/88; C12P 19/14; C12P 21/02

(52) U.S. Cl. CPC C12N 5/0075; B33Y 70/00; C08J 3/075; C12N 9/2474; C12N 9/6491; C12N 9/88; C12P 19/14; C12P 21/02; C12N 2533/30; C12N 2533/54; C12N 2533/72; C12N 2533/80; C12N 2537/10; C12N 2539/00; C12Y 302/01035; C12Y 302/01132; C12Y 304/24007; C12Y 402/02003

(57) ABSTRACT

The present disclose relates to a high-reactivity hydrogel particle and preparation method therefor and use thereof. The preparation method comprises the following steps: (1) performing polymerization reaction of double-bond modified polyethylene glycol, a modified natural polymer and an optional active functional monomer to obtain a gel; (2) smashing the gel to obtain a gel particle; and (3) performing enzymolysis reaction of the gel particle and an enzyme to obtain the hydrogel particle with reactivity. The double-bond modified polyethylene glycol has a double bond and a polyethylene glycol chain segment, the modified natural polymer has sulfydryl or a double bond, the active functional monomer is selected from a sulfydryl functionalized biological adhesive peptide or sulfydryl functionalized cholesterol, and the enzyme is an enzyme corresponding to the natural polymer. The obtained hydrogel particle has high reactivity and can be used for subsequent secondary crosslinking. The hydrogel particle also has good biocompatibility.

### TECHNICAL FIELD

The present disclose relates to the technical field of hydrogel particles, in particular to a high-reactivity hydrogel particle and a preparation method therefor and use thereof.

### BACKGROUND

A particle gel is a multi-functional material, which has wide application prospects. The particle gel has important application potential in the medical field due to its characteristics including injectability, self-healing and modularity. First, the injectability of the particle gel makes it become an ideal carrier for cell delivery and drug transportation. Through precise positioning and transportation via a syringe, the particle gel may be precisely delivered to a target position, so as to achieve effective treatment and delivery. Second, the particle gel has a self-healing ability, and can repair cracks or fractures by itself. Such feature increases the stability and prolongs the service life of the gel and reduces the damage and failure risks of a material. In addition, the particle gel also has the characteristic of modularization to conduct customization and combination as needed. This allows the constitutional units of the gel to be flexibly adjusted according to specific application demands, thereby meeting personalized requirements in the fields of cell delivery, tissue engineering, bioprinting and the like. In order to further improve the application performance of the particle gel, it is crucial to perform surface modification on hydrogel particles. By introducing a specific bioactive molecule or a surface modifier, the interaction ability of the gel and cells or tissues can be increased, and the biocompatibility and biological adhesiveness can be improved. In addition, customized mechanical properties are also vital. Different types of tissues and cells have different requirements on materials, and therefore the compositions, crosslinking degree and other factors of the gel particle need to be adjusted to achieve the customization of rigidity, elasticity and deformability, so as to meet the culture and growth demands of different types of tissues and cells. Hence, it is of great practical significance to develop large-scale preparation methods of high-reactivity hydrogel particles. Such methods are able to conveniently conduct surface modification and mutual crosslinking, thereby meeting the leading requirements of a biomedicine field on personalized demands of a new material carrier. With the deep understanding and continuous innovation of particle gel performances, this material will bring more innovation applications for cell delivery, tissue engineering, bioprinting and other fields.

The traditional modification methods of active hydrogel particles include a micro-droplet self-assembly method, a layer-by-layer chemical assembly method, a chemical grafting method, etc. Although these methods have strong controllability, hydrogel particles with good monodispersity may be obtained, however, the obtained hydrogel particles are low in flux and cannot be produced in large scales. In addition, these methods are tedious in purification steps and extremely high in production cost due to use of surfactants, dispersed oil phases and the like.

For example, Chinese patent CN112458075A discloses a double cross-linked particle gel. The preparation method of the double cross-linked particle gel includes the following steps: (1) preparing a precursor solution of a polyvinyl polyethylene glycol polymer, thiolated sodium alginate and microorganisms; (2) preparing hydrogel microspheres encapsulated with microorganisms, and preparing the microspheres through a water-in-oil emulsion dispersion method and solidifying the prepared microspheres; and (3) preparing a particle gel filling column with a double cross-linked structure for biocatalytic reaction to enhance a microbial catalysis process. The preparation method involves the water-in-oil emulsion dispersion method which has the disadvantages that the process is complex and cannot be used for large-scale production, and the obtained double cross-linked particle gel has extremely low reactivity and cannot be used for subsequent secondary crosslinking.

For another example, Chinese patent CN114773608A discloses a long-acting hyaluronic acid for treating osteoarthritis. The long-acting hyaluronic acid is obtained by co-blending modification of a branched macromolecule, a thiolated hyaluronic acid and a solvent. Wherein, although the branched macromolecule can be grafted to the skeleton of the thiolated hyaluronic acid, the obtained modified hyaluronic acid has extremely low reactivity and cannot be used for subsequent secondary crosslinking. Furthermore, the preparation process involves a dialysis step which needs to use a large amount of dialysis solution, making large-scale production difficult.

### SUMMARY

The objective of the present disclose is to prepare a high-reactivity hydrogel particle. The preparation process is simple and can achieve large-scale production, and the obtained hydrogel particle has high reactivity and can be used for subsequent secondary crosslinking. The hydrogel particle also has good biocompatibility.

In order to achieve the above objective, the technical solution adopted by the present disclose is as follows:

A preparation method of a hydrogel particle with reactivity, the preparation method including the following steps:
(1) performing polymerization reaction of double-bond modified polyethylene glycol, a modified natural polymer and an optional active functional monomer to obtain a gel;
(2) smashing the gel to obtain a gel particle; and
(3) performing enzymolysis reaction of the gel particle and an enzyme to obtain the hydrogel particle with reactivity;

The double-bond modified polyethylene glycol has a double bond and a polyethylene glycol chain segment, the modified natural polymer has sulfydryl or a double bond, the active functional monomer is selected from a sulfydryl or double-bond functionalized biological adhesive peptide or sulfydryl or double-bond functionalized cholesterol, and the enzyme is an enzyme corresponding to the natural polymer.

In the present disclose, the optional active functional monomer means that the active functional monomer can be added or cannot be added in step (1). When the active functional monomer is not added, the double-bond modified polyethylene glycol and the modified natural polymer are subjected to polymerization reaction. When the active functional monomer is added, the double-bond modified polyethylene glycol, the modified natural polymer and the active functional monomer are subjected to polymerization reaction.

In the present disclose, the gel obtained in step (1) has incompletely reacted double bonds or other active groups, the gel is in a crosslinking state, and most of the incompletely reacted double bonds or other active groups are located inside the cross-linked structure of the gel. In step (3), an enzyme corresponding to the natural polymer is used to perform enzymolysis and cutting on the natural polymer chain segment, which makes the incompletely reacted double bonds or other active groups in step (1) exposed onto the surface of the hydrogel particle, so that the hydrogel particle has high reactivity. This high reactivity facilitates subsequent applications of the hydrogel particle, for example cell culture, or secondary crosslinking is performed by utilizing such high reactivity.

In some embodiments, the double-bond modified polyethylene glycol is selected from one or more of the group consisting of polyethylene glycol monoacrylate, polyethylene glycol diacrylate, polyethylene glycol monomethacrylate, polyethylene glycol dimethacrylate, hyperbranched polyethylene glycol diacrylate and hyperbranched poly(β-hydrazide ester).

In some embodiments, the hyperbranched polyethylene glycol diacrylate is obtained by live polymerization of polyethylene glycol diacrylate.

In some embodiments, the hyperbranched polyethylene glycol diacrylate is obtained by reversible addition-fragmentation chain transfer (RAFT) live polymerization of polyethylene glycol diacrylate.

In some embodiments, the chain transfer agent used in the RAFT live polymerization is

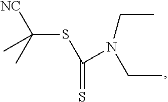

and the structural formula of the polyethylene glycol diacrylate is

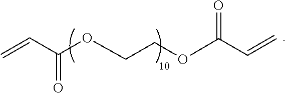

In some embodiments, the structural formula of the hyperbranched polyethylene glycol diacrylate is:

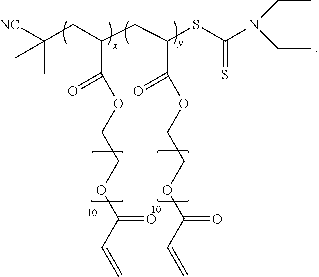

In some embodiments, the hyperbranched poly(β-hydrazide ester) is obtained by Michael addition reaction of 3,3′-dithiodipropionyl hydrazine with primary amine group and polyethylene glycol diacrylate.

In some embodiments, the structural formula of the hyperbranched poly(β-hydrazide ester)

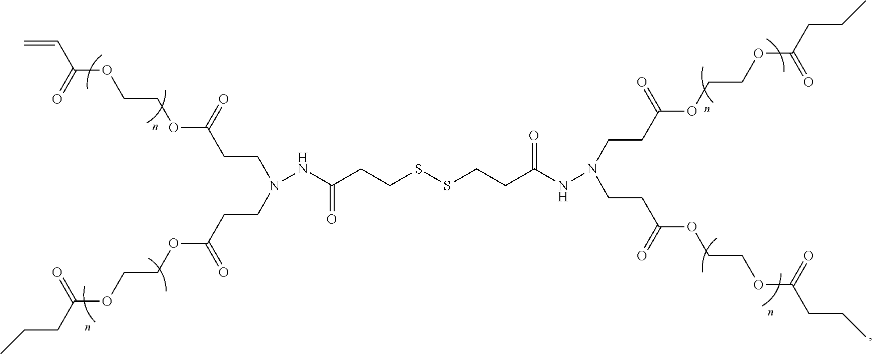

wherein, n is 5-20, preferably, 10. The hyperbranched poly(β-hydrazide ester) contains a disulfide bond, which has relatively small positive charges, and cells are slightly negatively charged. When the hyperbranched poly(β-hydrazide ester) is used as a polymerization monomer in step (1), the finally obtained hydrogel particle has a better cell adhesion effect when being subsequently used in cell culture and other fields.

In some embodiments, the natural polymer is selected from one or more of the group consisting of collagen, chitosan, gelatin, glucan, sodium alginate and hyaluronic acid.

In some embodiments, the biological adhesive peptide is a small molecule peptide containing sequence fragments such as arginine-glycine-aspartic acid-cysteine, mercaptopropionic acid-arginine-glycine-aspartic acid, or glycine-arginine-glycine-aspartic acid-serine-proline-cysteine.

In some embodiments, the sulfydryl functionalized biological adhesive peptide is sulfydryl functionalized arginine-glycine-aspartic acid (RGD) with amino acid series of GRGDSPC.

In some embodiments, the polymerization reaction in step (1) is carried out in water or phosphate buffer saline (PBS), the mass/volume concentration of the double-bond modified polyethylene glycol in water or PBS is 2-20%, the mass/volume concentration of the modified natural polymer in water or PBS is 0.5-20%, and the mass/volume concentration of the active functional monomer in water or PBS is 0-20%.

In some embodiments, a mass ratio of the double-bond modified polyethylene glycol to the modified natural polymer to the active functional monomer is 5:1.5:0.5.

In some embodiments, the modified natural polymer has sulfydryl, the polymerization reaction is Michael addition polymerization, and the temperature of the polymerization reaction is 0-30° C.

In some embodiments, the modified natural polymer has a double bond, the polymerization reaction is free radical polymerization, and the polymerization reaction is carried out in the presence of a photoinitiator.

In some embodiments, the smashing in step (2) is selected from attrition crushing, grinding crushing or impact crushing.

Preferably, the smashing in step (2) is grinding crushing.

Further, the grinding and crushing frequency is 1-5 times. The more the grinding frequency, the smaller the particle size of the finally obtained hydrogel particle.

Further, the duration for the grinding crushing each time is 1-10 min, preferably 5 min.

In some embodiments, the particle size of the hydrogel particle with reactivity is 30 μm-300 μm.

Preferably, the particle size of the hydrogel particle with reactivity is 100 μm-150 μm.

In some embodiments, the polymerization reaction in step (1) is carried out at pH of 7-8.

In some embodiments, the concentration of the enzyme in step (3) is 5 U/mL-2000 U/mL.

Preferably, the polymerization reaction in step (1) is carried out at pH of 7.2-7.4.

In some embodiments, the enzyme in step (3) is selected from one or more of the group consisting of hyaluronidase, sodium alginate lyase, pancreatin, chitosanase or collagenase.

The present disclose also provides the hydrogel particle with reactivity obtained by the above preparation method of the hydrogel particle with reactivity. The hydrogel particle has high reactivity, and can be used for subsequent secondary crosslinking. The hydrogel particle also has good biocompatibility.

The present disclose also provides use of the foregoing hydrogel particle with reactivity in cell culture, tissue engineering, biological scaffolds, drug delivery, or bioprinting.

The hydrogel particle of the present disclose uses the double-bond modified polyethylene glycol and the modified natural polymer having sulfydryl or a double bond as main polymerization monomers, specifically, a gel block is prepared by polymerization and then smashed to obtain the gel particle, the active sites of the gel particle are exposed by utilizing enzyme treatment to finally obtain the high-reactivity hydrogel particle. The biological molecule, drugs or cells can more easily interact with the active sites so as to enhance the availability of activity; the reactive sites can provide support for cell adhesion, thereby promoting adhesion and proliferation of cells; the reactive sites can also provide binding sites or target receptors for more drugs, thereby improving the delivery effect and effectiveness of drugs.

Due to use of the above technical solution, the present disclose has the following advantages compared with the prior art:
(1) High reactivity: the gel obtained in step (1) in the present disclose has incompletely reacted active double bonds or other introduced active groups, the gel is in a cross-linked state, most of the incompletely reacted active double bonds or other active groups are located inside the cross-linked structure or coated with the natural polymer without effective exposure on the surface of the hydrogel, and the use of the enzyme corresponding to the natural polymer in step (3) to perform enzymolysis and cutting on the natural polymer chain segment makes the incompletely reacted double bonds or other active groups in step (1) exposed on the surface of the hydrogel particle, so that the hydrogel particle has high chemical reactivity and meanwhile the exposure of a large amount of active groups will greatly enhance the interaction between viable cells and the surface of the material and promote early adhesion and proliferation of the cells on the surface of the material. In addition, the above active double bonds can subsequently perform secondary crosslinking with the modified polymer, and the obtained secondary cross-linked gel particle can increase the density of the cross-linked network inside the particle gel and affect the pore structure and pore size distribution of the particle gel, thereby regulating the release rate of the drug or the bioactive substance.

The double-bond modified polyethylene glycol can effectively regulate the mechanical properties of the hydrogel particle. The double-bond modified polyethylene glycol can also avoid quick degradation of the gel particle in step (3) during enzymolysis, thereby effectively promoting the exposure of the double bonds and other active groups.
(2) The preparation process is simple, and can achieve batch and low-cost preparation of the high-reactivity hydrogel particle. When similar hydrogel particles are prepared by using the existing technology, complex processes such as the water-in-oil emulsion dispersion method need to be used. These processes are all not beneficial for large-scale batch production, whereas the present disclose adopts simple smashing and enzymolysis processes so as to obtain the high-reactivity hydrogel particle, with mild and green preparation processes and no complex equipment or operations. The preparation method of the present disclose does not involve the use of surfactants or oil phases, which not only is simple in process but also greatly reduces the subsequent purification cost.
(3) The obtained hydrogel particle has good biocompatibility. The raw materials for preparing the hydrogel particle in the present disclose are safe, non-toxic and high in biocompatibility, the obtained hydrogel particle has application prospects in the fields of cell culture, tissue engineering, drug delivery, bioprinting and the like.

### BRIEF DESCRIPTION OF THE DRAWINGS

FIG. 1 is ¹H-NMR spectrogram of hyperbranched polyethylene glycol diacrylate;

FIG. 2 is ¹H-NMR spectrogram of hyperbranched poly(β-hydrazide ester);

FIG. 3 is ¹H-NMR spectrogram of hyaluronic acid and thiolated hyaluronic acid, wherein HA is hyaluronic acid, and HA-SH is thiolated hyaluronic acid;

FIG. 4 is a preparation flowchart of a hydrogel particle according to the present disclose;

FIG. 5 is a scanning electron microscopy (SEM) image of a hydrogel particle prepared in example 1;

FIG. 6 is a microscopy image and size distribution of hydrogel particles with different grinding times in examples 1-3;

FIG. 7 is a cell-hydrogel adhesive growth graph after hyaluronidase treatment is carried out for 12 h in example 1 and comparative example 1;

FIG. 8 is a cell adhesion and proliferation graph in example 8 and comparative example 2.

FIG. 9 is a physical picture of a hydrogel particle before and after secondary crosslinking after hyaluronidase treatment is carried out for 12 h in example 8;

FIG. 10 shows strain-modulus curves before and after secondary crosslinking of a hydrogel particle after hyaluronidase treatment is carried out for 12 h in example 1;

FIG. 11 shows strain-modulus curves of a hydrogel particle in example 10 and a control particle;

FIG. 12 shows degradation curves of a hydrogel particle in example 11 and a control particle under the condition of 500 U/mL hyaluronidase;

FIG. 13 is a biocompatibility-live dead cell staining diagram of a 5 mg/mL active hydrogel material extraction solution in example 1.

### DETAILED DESCRIPTION OF THE EMBODIMENTS

Next, the technical solution of the present disclose will be further illustrated in combination with accompanying drawings.

### Preparation Example 1

### Preparation of Hyperbranched Polyethylene Glycol Diacrylate (HB-PEGDA):

RAFT polymerization was carried out by using DS (with a structural formula was

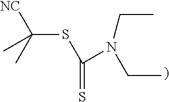

as an RAFT reagent, 2′-azodi(2-methylnitrile) (AIBN) as an initiator and homopolymer polyethylene glycol diacrylate (PEGDA) (with a structural formula of

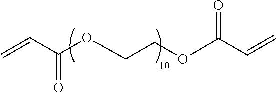

(average Mn=575) (0.4 mol·L⁻¹) as a monomer in butanone at 70° C.; a molar ratio of [PEGDA]:[DS]:[AIBN] was 25:1:1.4, a polymer product HB-PEGDA was obtained, its structural formula was

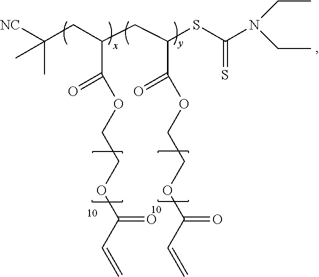

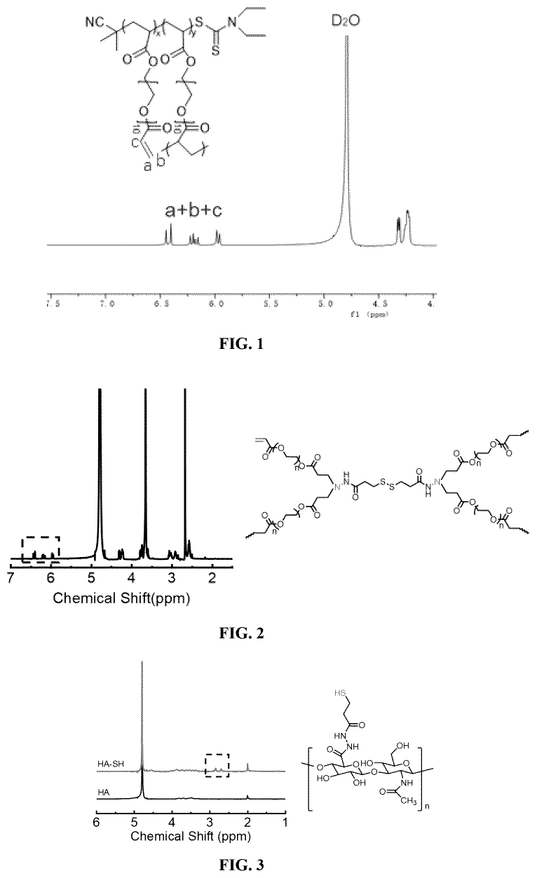

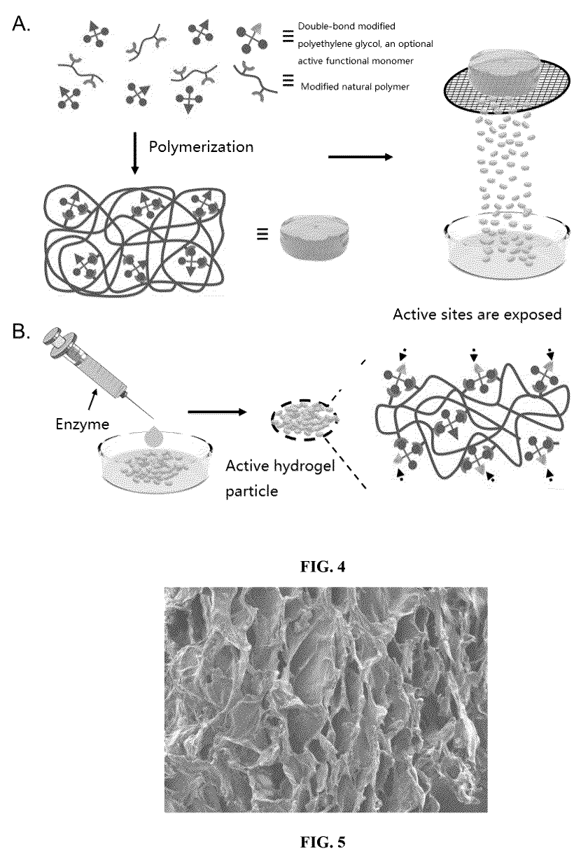

and its ¹H NMR spectrogram is as shown in FIG. 1.

### Preparation Example 2

### Preparation of Hyperbranched Poly(β-Hydrazide) Ester (HB-PBHE):

1) PEGDA and 3,3′-dithiobis(propionohydrazide) (DTP) were dissolved into dimethyl sulfoxide (DMSO) in a molar ratio of 5:2 at 90° C. for reaction. The structure of the obtained HB-PBHE is

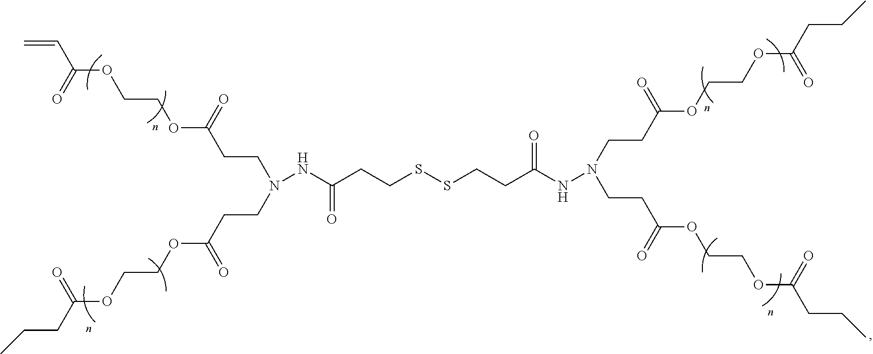

and its ¹H NMR spectrogram is as shown in FIG. 2.

### Example 1

30 mg of HA-SH (its ¹H NMR spectrogram is as shown in FIG. 3) was dissolved into 1 mL of deionized water to prepare a 3% (mass/volume concentration w/v) precursor solution A; 100 mg of HB-PBHE obtained in preparation example 2 was dissolved into 1 mL of deionized water to prepare a 10% (mass/volume concentration w/v) precursor solution B. The pH of the precursor solution A was adjusted to 7.2 using a sodium hydroxide aqueous solution, and subsequently the adjusted precursor solution A and the precursor solution B were evenly mixed in a volume ratio of 1:1 and then subjected to standing so as to obtain a hydrogel. The above prepared hydrogel was ground for 5 min by using a 100-meshed metal screen and then treated for 12 h with a 500 U/mL hyaluronidase to obtain an active hydrogel particle with a particle size of 100 μm. The SEM image of the obtained active hydrogel particle is as shown in FIG. 5, and the particle size graph of the obtained active hydrogel particle is a graph corresponding to the first grinding in FIG. 6.

### Example 2

This example is basically the same as example 1 only except that grinding was carried out twice, the duration of grinding each time was the same as that in example 1, and the particle size of the finally obtained active hydrogel particle was 70 μm-80 μm. The particle size graph of the finally obtained active hydrogel particle is a graph corresponding to the second grinding in FIG. 6.

### Example 3

This example is basically the same as example 1 only except that grinding was carried out three times, the duration of grinding each time was the same as that in example 1, and the particle size of the finally obtained active hydrogel particle was 50 μm-60 μm. The particle size graph of the finally obtained active hydrogel particle is a graph corresponding to the third grinding in FIG. 6.

It can be seen that as the grinding times increases, the diameter of the hydrogel particle continuously decreases.

### Example 4

20 mg of thiolated sodium alginate was dissolved into 1 mL of deionized water to prepare a 2% (mass/volume concentration w/v) precursor solution A; 100 mg of HB-PEGDA obtained in preparation example 1 was dissolved into 1 mL of deionized water to prepare a 10% (mass/volume concentration w/v) precursor solution B. The pH of the precursor solution A was adjusted to 7.3, and the adjusted precursor solution A and the precursor solution B were evenly mixed in a volume ratio of 2:1 and then subjected to standing so as to obtain a hydrogel. The above prepared hydrogel was ground for 5 min by using a 100-meshed metal screen and then treated for 12 h with a 500 U/mL sodium alginate lyase to obtain an active hydrogel particle with particle size distribution of 100 μm-150 μm.

### Example 5

20 mg of thiolated gelatin (Gelatin-SH) was dissolved into 1 mL of deionized water to prepare a 2% (mass/volume concentration w/v) precursor solution A; 100 mg of HB-PEGDA obtained in preparation example 1 was dissolved into 1 mL of deionized water to prepare a 10% (mass/volume concentration w/v) precursor solution B. The pH of the precursor solution A was adjusted to 7.4, and the adjusted precursor solution A and the precursor solution B were evenly mixed in a volume ratio of 2:1 and then subjected to standing so as to obtain a hydrogel. The above prepared hydrogel was ground for 5 min by using a 100-meshed metal screen and then treated for 12 h with a 500 U/mL collagenase to obtain an active hydrogel particle with particle size distribution of 100 μm-150 μm.

### Example 6

20 mg of thiolated chitosan SH-chitosan (SH-CS) was dissolved into 1 mL of deionized water to prepare a 2% (mass/volume concentration w/v) precursor solution A; 100 mg of HB-PBHE obtained in preparation example 2 was dissolved into 1 mL of deionized water to prepare a 10% (mass/volume concentration w/v) precursor solution B. The pH of the precursor solution A was adjusted to 7.4, and the adjusted precursor solution A and the precursor solution B were evenly mixed in a volume ratio of 2:1 and then subjected to standing so as to obtain a hydrogel. The above prepared hydrogel was ground for 5 min by using a 100-mesh metal screen, and then treated for 12 h with a 500 U/mL chitosanase to obtain an active hydrogel particle with particle size distribution of 100 μm-150 μm.

### Comparative Example 1

This comparative example is basically the same as that in example 1 only except that the step of treatment for 12 h with a 500 U/mL hyaluronidase was not carried out, that is, the hydrogel particle was directly obtained after grinding.

### Example 7

The active hydrogel particle prepared in example 1 and the hydrogel particle prepared in comparative example 1 were secondarily crosslinked with 1 mg/mL sulfydryl polyethylene glycol rhodamine B, a mass ratio of the hydrogel particles to the sulfydryl polyethylene glycol rhodamine B was 500:1, and the above materials were incubated for 30 min. After incubation was ended, the obtained product was washed with PBS and then observed under the fluorescence microscope. The results are as shown in FIG. 7. In FIG. 7, the enzymolysis in the left panel and 3 h of enzymolysis in the right panel indicate the results corresponding to example 1, and untreated in the left panel and 0 h of enzymolysis in the right panel in FIG. 7 indicate the results corresponding to comparative example 1, from which it can be seen that the active functional group of the hydrogel particle after being subjected to enzyme treatment in step (3) can be effectively exposed, the obtained hydrogel particle has high reactivity and subsequently can react with a sulfydryl-containing small molecule or large molecule and the like, and the gel particle without enzyme treatment has extremely low reactivity.

### Example 8

30 mg of HA-SH was dissolved into 1 mL of deionized water to prepare a 3% (mass/volume concentration w/v) precursor solution A; 100 mg of hyperbranched poly(β-hydrazide) ester obtained in preparation example 2 and 10 mg of thiolated adhesive peptide RGD-SH (the amino acid series was GRGDSPC) were dissolved into 1 mL of deionized water to prepare a precursor solution B. The pH of the precursor solution A was adjusted to 7.4, then the adjusted precursor solution A and the precursor solution B were evenly mixed in a volume ratio of 1:1, and the hydrogel was finally obtained after standing. The above prepared hydrogel was ground for 5 min by using a 100-mesh metal screen, treated for 12 h with a 500 U/mL hyaluronidase, and then washed for 3 times with PBS to obtain an active hydrogel particle. The active hydrogel particle was soaked in MEM (Minimum Essential Medium) complete culture medium for 1 h, and then 5000 L929 cells were added, a culture plate was placed in an incubator for culture, and the growth state of cells was observed. The results of culture for 48 h, 72 h and 96 h are as shown in pictures of enzyme treatment in FIG. 8, from which it can be seen that the active groups of the active hydrogel particle obtained after enzyme treatment are exposed, which can effectively allow cells to be adhered and proliferated.

### Comparative Example 2

This comparative example was basically the same as example 8 only except that the step of treatment for 12 h with a 500 U/mL hyaluronidase was not carried out. That is, the hydrogel particle was directly obtained after grinding. The results of cell culture for 24 h, 36 h and 48 h are as shown in pictures showing “untreated” in FIG. 8, from which it can be seen that when the step of enzyme treatment in the present disclose is used, the hydrogel particle has higher reactivity; when use in cell culture, the cell adhesion and proliferation effects are better.

### Example 9

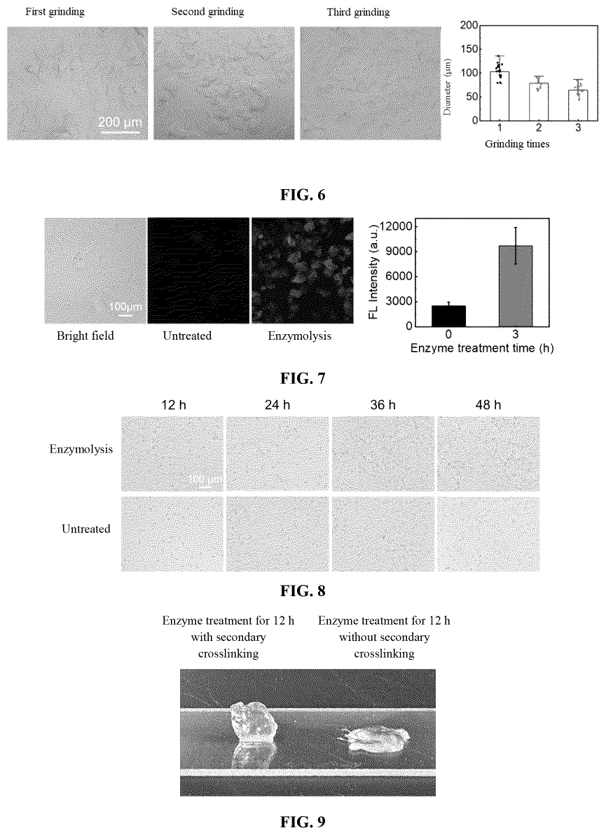

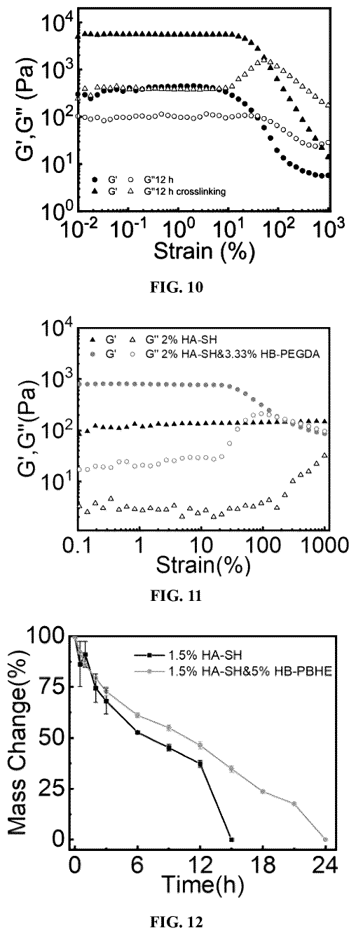

The active hydrogel particle prepared in example 1 was mixed with HA-SH for secondary crosslinking. The results are as shown in the left panel of FIG. 9, from which it can be seen that the hydrogel particle after secondary crosslinking has large strength because HA-SH can react with the active functional groups exposed from the surface of the active hydrogel particle again. The right panel in FIG. 9 shows that the active hydrogel particle obtained in example 1 is not subjected to secondary crosslinking, from which it can be seen that the hydrogel particle has slightly low strength. Before and after secondary crosslinking, the storage modulus of the hydrogel particle is as shown in FIG. 10, from which it can be seen that the storage modulus after second crosslinking increases by two orders of magnitude. Therefore, the high-reactivity hydrogel particle prepared in the present disclose can have improved mechanical properties through subsequent secondary crosslinking, and has wide application prospects in the fields of tissue engineering, biological scaffolds, drug delivery carriers, bioprinting and the like.

### Example 10

This example was basically the same as example 4 only except that the mass/volume concentration w/v of the precursor solution B was replaced as 3.33%. The mechanical properties of the obtained gel particle are shown by 2% HA-SH&3.33% HB-PEGDA in FIG. 11.

By contrast, the mechanical properties of the corresponding gel particle are shown by 2% HA-SH in FIG. 11 when 2% HA-SH was used alone and HB-PEGDA was not added.

It can be seen that HB-PEGDA functions as a crosslinking agent, thereby effectively improving the mechanical properties of the hydrogel particle.

### Example 11

This example was basically the same as example 1 only except that the mass/volume concentration w/v of the precursor solution A was replaced as 1.5%, and the mass/volume concentration w/v of the precursor solution B was replaced as 5%. The degradation curve of the obtained gel particle was shown by 1.5% HA-SH % &5% HB-PBHE in FIG. 12.

By contrast, the degradation curve of the corresponding gel particle is shown by 1.5% HA-SH in FIG. 12 when 1.5% HA-SH was used alone and HB-PBHE was not added.

It can be seen that the hydrogel particle without PBHE is more quickly degraded, and therefore the addition of PBHE can not only provide additional reaction sites but also avoid the too-quick degradation of the hydrogel particle during enzymolysis, thereby effectively promoting the exposure of the double bond and the active groups.

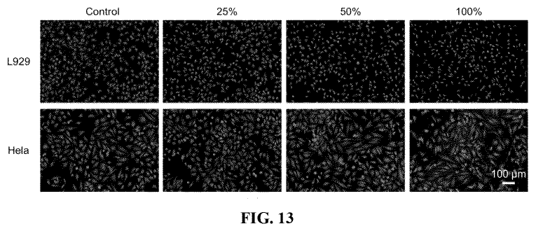

FIG. 13 shows the results for biocompatibility test of a 5 mg/mL hydrogel extract after the hydrogel particle prepared in example 1 is freeze-dried for 3 d. It can be seen from FIG. 13 that almost all the cells are green (viable cells), proving that the hydrogel particle of the present disclose has good biocompatibility.

1. A preparation method of a hydrogel particle with reactivity, wherein the preparation method comprising the following steps:
(1) performing polymerization reaction of double-bond modified polyethylene glycol, a modified natural polymer and an optional active functional monomer to obtain a gel;
(2) smashing the gel to obtain a gel particle; and
(3) performing enzymolysis reaction of the gel particle and an enzyme to obtain the hydrogel particle with reactivity;
wherein the double-bond modified polyethylene glycol has a double bond and a polyethylene glycol chain segment, the modified natural polymer has sulfydryl or a double bond, the active functional monomer is selected from a sulfydryl or double-bond functionalized biological adhesive peptide or sulfydryl or double-bond functionalized cholesterol, and the enzyme is an enzyme corresponding to the natural polymer.

2. The preparation method of the hydrogel particle with reactivity according to claim 1, wherein the double-bond modified polyethylene glycol is selected from one or more of the group consisting of polyethylene glycol monoacrylate, polyethylene glycol diacrylate, polyethylene glycol monomethacrylate, polyethylene glycol dimethacrylate, hyperbranched polyethylene glycol diacrylate and hyperbranched poly(β-hydrazide ester), the hyperbranched polyethylene glycol diacrylate is obtained by live polymerization of polyethylene glycol diacrylate; and the hyperbranched poly(β-hydrazide ester) is obtained by Michael addition reaction of 3,3′-dithiodipropionyl hydrazine with primary amine group and polyethylene glycol diacrylate.

3. The preparation method of the hydrogel particle with reactivity according to claim 2, wherein the hyperbranched polyethylene glycol diacrylate is obtained by reversible addition-fragmentation chain transfer (RAFT) live polymerization of polyethylene glycol diacrylate.

4. The preparation method of the hydrogel particle with reactivity according to claim 3, wherein the chain transfer agent used in the RAFT live polymerization is 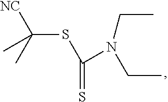
and the structural formula of the polyethylene glycol diacrylate is

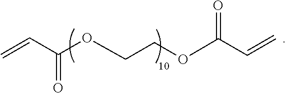

5. The preparation method of the hydrogel particle with reactivity according to claim 2, wherein the structural formula of the hyperbranched polyethylene glycol diacrylate is: 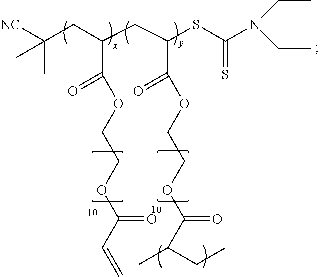
and/or, the structural formula of the hyperbranched poly(β-hydrazide ester) is 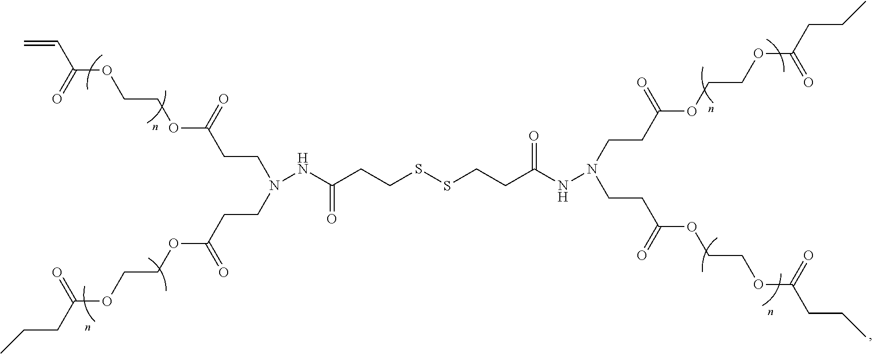
wherein, n is 5-20.

6. The preparation method of the hydrogel particle with reactivity according to claim 1, wherein the natural polymer is selected from one or more of the group consisting of collagen, chitosan, gelatin, glucan, sodium alginate and hyaluronic acid.

7. The preparation method of the hydrogel particle with reactivity according to claim 1, wherein the polymerization reaction in step (1) is carried out in water or phosphate buffered saline (PBS), the mass/volume concentration of the double-bond modified polyethylene glycol in water or PBS is 2-20%, the mass/volume concentration of the modified natural polymer in water or PBS is 0.5-20%, and the mass/volume concentration of the active functional monomer in water or PBS is 0-20%.

8. The preparation method of the hydrogel particle with reactivity according to claim 1, wherein the modified natural polymer has sulfydryl, the polymerization reaction is Michael addition polymerization, and the temperature of the polymerization reaction is 0-30° C.; or, the modified natural polymer has a double bond, the polymerization reaction is free radical polymerization, and the polymerization reaction is carried out in the presence of a photoinitiator.

9. The preparation method of the hydrogel particle with reactivity according to claim 1, wherein smashing in step (2) is selected from attrition crushing, grinding crushing or impact crushing; and/or, the particle size of the hydrogel particle with reactivity is 30 μm-300 μm.

10. The preparation method of the hydrogel particle with reactivity according to claim 1, wherein the polymerization reaction in step (1) is carried out at pH of 7-8; and/or, the concentration of the enzyme in step (3) is 5 U/mL-2000 U/mL; and/or, the enzyme in step (3) is selected from one or more of the group consisting of hyaluronidase, sodium alginate lyase, pancreatin, chitosanase or collagenase.

11. The hydrogel particle with reactivity obtained by the preparation method of the hydrogel particle with reactivity according to claim 1.

12. (canceled)

13. A cell culture medium, wherein, the cell culture medium comprising the hydrogel particle with reactivity according to claim 11.

14. A tissue engineering material, wherein, the tissue engineering material comprising the hydrogel particle with reactivity according to claim 11.

15. A biological scaffold material, wherein, the biological scaffold material comprising the hydrogel particle with reactivity according to claim 11.

16. A drug delivery material, wherein, the drug delivery material comprising the hydrogel particle with reactivity according to claim 11.

17. A bioprinting material, wherein, the bioprinting material comprising the hydrogel particle with reactivity according to claim 11.
